SEQUENCE LISTING (5 sequences)

SEQ ID NO: 1 - protein, 165 aa, Artificial
  FcRgamma IIB immune system receptor minimal therapuetic protein sequence
AVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITV

SEQ ID NO: 2 - protein, 165 aa, Artificial
  FcRgamma III immune system receptor minimal therapuetic protein sequence
AVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITI

SEQ ID NO: 3 - protein, 185 aa, Artificial
  FcRgamma IIB immune system receptor minimal therapuetic protein sequence extended at the termini by wild-type protein sequence containing parts of signal and/or linker
MGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGII

SEQ ID NO: 4 - protein, 176 aa, Artificial
  FcRgamma III immune system receptor minimal therapuetic protein sequence extended at the termini by wild-type protein sequence containing parts of signal and/or linker
MRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQG

SEQ ID NO: 5 - protein, 181 aa, Artificial
  FcRgamma IIA immune system receptor minimal therapuetic protein sequence
AAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSRLDPTFSIPQANHSHSGDYHCTGNIGYTLFSSKPVTITVQVPSMGSSSPM

(19) United States
(12) Patent Application Publication
(10) Pub. No.: US 2008/0214459 A1
(43) Pub. Date: Sep. 4, 2008

(54) Pharmaceutical composition containing sFcyRIIb

(75) Inventors: Robert Huber, Germering (DE); Peter Sondermann, Rudolfstetten (CH); Uwe Jacob, Muenchen (DE)

Correspondence Address: ROTHWELL, FIGG, ERNST & MANBECK, P.C., 1425 K STREET, N.W., SUITE 800, WASHINGTON, DC, 20005, US

(73) Assignee: Max-Planck-Gesellschaft zur Foerderung der Wissenschaften E.V., Muenchen (DE)

(21) Appl. No.: 11/971,438

(22) Filed: Jan. 9, 2008

Related U.S. Application Data
(63) Continuation of application No. 10/851,655, filed on May 24, 2004, now abandoned.
Application No. 10/851,655 is a continuation-in-part of application No. PCT/EP2002/013080, filed on Nov. 21, 2002.

(30) Foreign Application Priority Data
Nov. 22, 2001 (DE) 101 57 290 5 (DE)

(51) Int. Cl. A61K 38/00; A61P 37/00

(52) U.S. Cl. 514/12

(57) ABSTRACT

The present invention concerns pharmaceutical compositions containing one of the receptors FcγR IIa, FcγR IIb or FcγR III in a recombinantly produced, soluble form and their use to treat diseases or conditions which are caused by overshooting immune reactions and a pathologically increased formation of antibodies, in particular of autoantibodies. Multiple sclerosis, systemic lupus erythematosus and rheumatoid arthritis are particularly important fields of application.

This application is a continuation of U.S. Ser. No. 10/851,655 filed May 24, 2004, which is a continuation in part of PCT/EP2002/013080 filed Nov. 21, 2002.

The present invention concerns pharmaceutical compositions that contain one of the receptors FcγR IIa, FcγR IIb or FcγR III in a recombinantly produced, soluble form.

Fc receptors (FcRs) play an important role in defence reactions of the immune system. When pathogens have entered the blood circulation they are bound by immunoglobulins, the antibodies. Due to the multivalency of the Fc fragments of the antibodies, the resulting immune complexes bind with high avidity to Fc receptor-presenting phagocytes which destroy and eliminate the pathogens. Accessory cells such as natural killer cells, eosinophils and mast cells also carry Fc receptors on their surface which release stored mediators such as growth factors or toxins after binding of immune complexes that support the immune response.

Hence the Fc receptors of the accessory cells are signal molecules and specifically bind immunoglobulins of various isotypes during the humoral immune response. In addition Fc receptors can activate natural killer cells to destroy antibody-coated target cells (“antibody-dependent cell-mediated cytotoxicity”, ADCC).

However, in addition to the positive effects of FcRs as a defence against pathogens, overshooting reactions may also occur which result in an undesired stimulation of the immune system in healthy persons that manifests itself especially as allergies or autoimmune diseases. Such immune reactions directed against the body's own substances remain a major medical problem and although there are approaches for treating them, they are not equally effective in every patient.

Medical problems are also associated with presence of elevated concentrations of natural killer cells (NK cells) which are also formed as a result of overshooting immune reactions. Thus it has been observed that miscarriages frequently occur in pregnant women with an elevated level of NK cells because it hinders the implantation as well as intrauterine growth of the foetus. High doses of immunoglobulins have been previously administered intravenously to treat pregnant women with elevated levels of NK cells. This intravenously administered immunoglobulin G (IVIg) is intended to neutralize NK cells and attenuate the overshooting immune response. IVIg has also been administered for autoimmune diseases (e.g. multiple sclerosis). However, the administration of IVIg in the high doses that are required causes considerable problems. In order to achieve an adequate IVIg concentration in the serum, it is necessary to infuse large amounts of this protein (25-150 g) which is carried out using a relatively large volume (250-1500 ml). This large amount of liquid has to be infused for 2 to 4 hours; a more rapid administration intensifies the side effects that are observed anyway such as headache, fever, general unweliness etc. It has to be administered on 1 to 3 consecutive days and the treatment has to be carried out once every month.

In addition to the considerable amount of time required and the side effects of such an IVIg treatment, another disadvantage is that the treatment is very expensive and costs about US $ 10,000 during a single pregnancy. Moreover, the treatment costs usually have to be borne by the patient herself. An IVIg treatment using a corresponding dosage for e.g. multiple sclerosis or other neurological diseases (Achiron et al., Neurology (1998), 50(2): 398-402; Dalakas, Ann. Intern. Med. (1977), 126(9): 721-30; Brannagan et al., Virology (1996), 47(3): 674-7) has the same disadvantages. WO 00/32767 describes soluble Fc receptors which are only composed of the extracellular part of the receptor and are not glycosylated. Due to the absence of the transmembrane domain and of the signal peptide, these proteins are present in a soluble form and not bound to cells. Furthermore the Fc receptors described in this document can be produced recombinantly and have been suggested for the treatment of autoimmune diseases since they can bind antibodies but do not have an effector effect on other components of the immune system. Hence they are able to neutralize antibodies in the bloodstream which has an attenuating effect especially on autoimmune processes. WO 00/32767 additionally describes the crystal structure of certain Fc receptors and the possibility of finding substances that inhibit the interaction of IgG with Fc receptors with the aid of these crystal structures. The elucidation of the crystal structure allows one to find such inhibitors by screening the available databases with the aid of computer programs.

The object of the present invention was to further develop the findings of WO 00/32767 and to provide treatment methods especially for the indications multiple sclerosis (MS), systemic lupus erythematosus (SLE) and rheumatoid arthritis (RA) and also for diseases with an elevated level of NK cells which avoid the disadvantages of the previous treatment methods, are easy to use and can be carried out cost-effectively.

This object was achieved within the scope of the present invention by pharmaceutical compositions in the form of aqueous solutions which contain recombinantly produced, soluble FcγR IIa, FcγR IIb FcγR IIb or FcγR III in an amount of 40 to 4000 mg, preferably 100 to 1000 mg and a concentration of up to 50 mg/ml (preferably e.g. 8 ml per injection). Within the scope of the present invention, it was found that in case that said receptors were produced recombinantly in prokaryotes and are therefore unglycosylated, i.e. should be poorly soluble, can nevertheless surprisingly be purified even with known methods in such a manner that a relatively high concentration of sFcR is obtained in a soluble form. Furthermore it was found that these receptors have exceptionally strong effects in combatting overshooting immune reactions even in relatively low doses and in particular at a dose of 0.5 mg/kg to 50 mg/kg body weight.

A preferred pharmaceutical composition, therefore, contains at least one soluble Fc receptor in an amount, which allows application of 0.5 to 50 mg receptor/kg body weight. Due to the availability of the receptors in a highly concentrated soluble form and based on the realization that relatively low amounts of the active sFcR are sufficiently effective, the pharmaceutical composition can be injected and thus laborious infusions lasting for several hours which is quite usual for IVIg treatments can be avoided. Moreover, the pharmaceutical composition according to the invention also generally contains excipients or/and auxiliary substances that are pharmaceutically acceptable and pharmacologically support or facilitate the use of the pharmaceutical composition.

The pharmaceutical composition preferably contains the FcγR IIb receptor which comprises at least the amino acid sequence shown in SEQ ID NO. 1 or the FcγR III receptor with the minimum sequence shown in SEQ ID NO. 2. These two sequences are minimal sequences which can in principle be extended at the termini with suitable sequences of the wild type proteins. It is preferable not to introduce mutations into the constructs when extending the N-termini or/and C-termini of the stated sequences in order to prevent antigenicity. However, it is theoretically possible to also introduce mutations or deletions into the extended sequences provided that they do not result in an undesired antigenicity.

Examples of sequences extended at the termini containing parts of signal or/and linker sequences which, although belonging to the gene, are not absolutely necessary for a therapeutic protein are SEQ ID NO. 3 for FcγR IIb and SEQ ID NO. 4 for FcγR III.

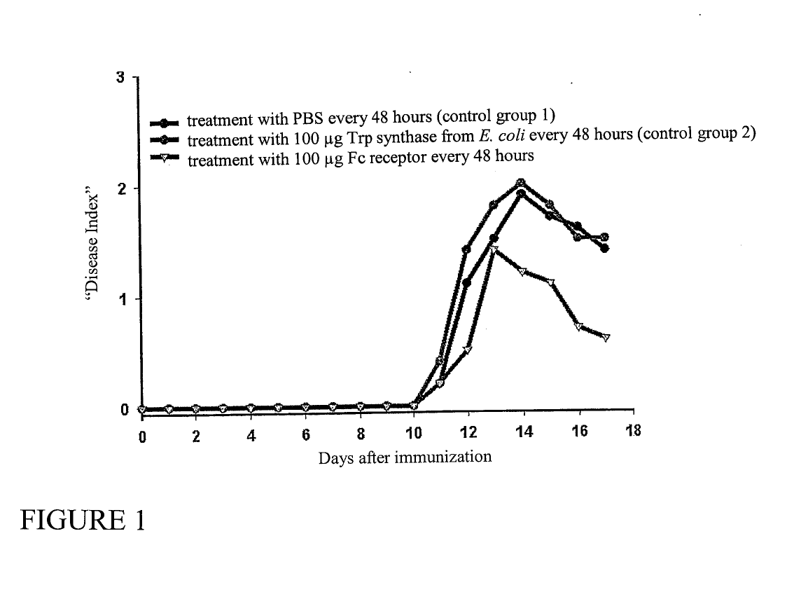

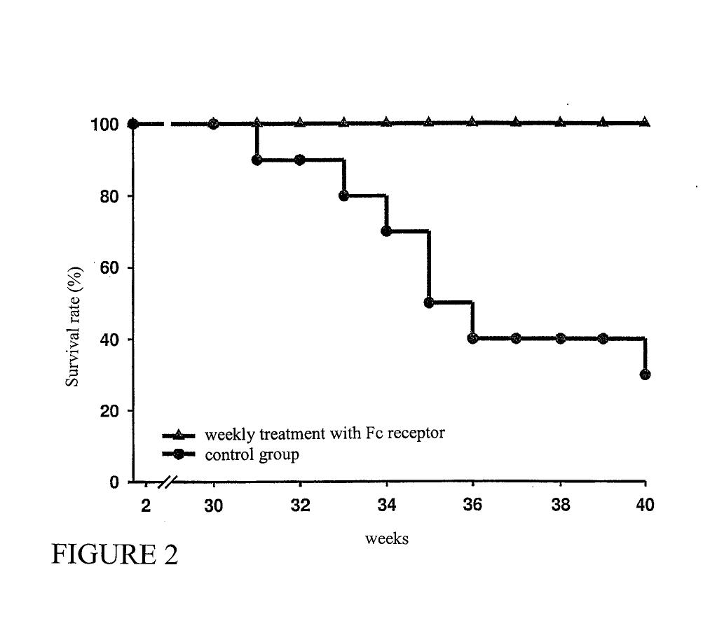

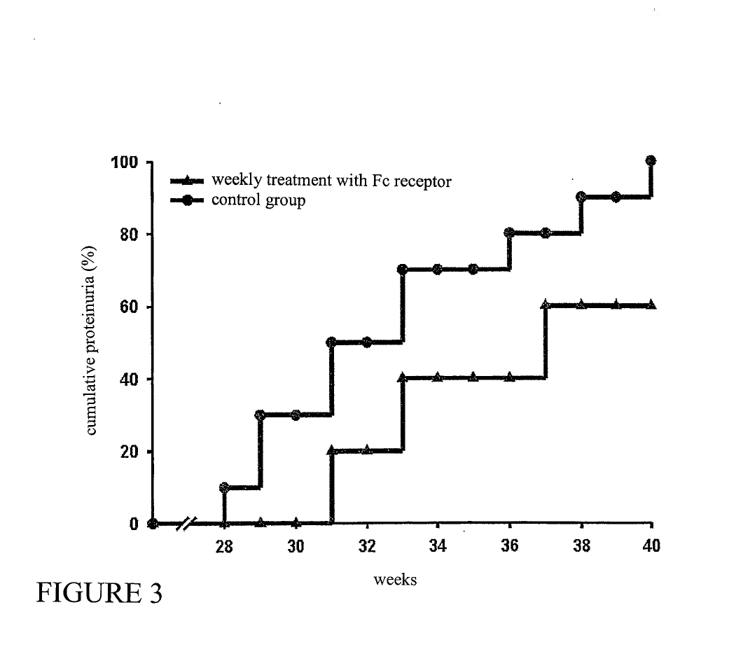

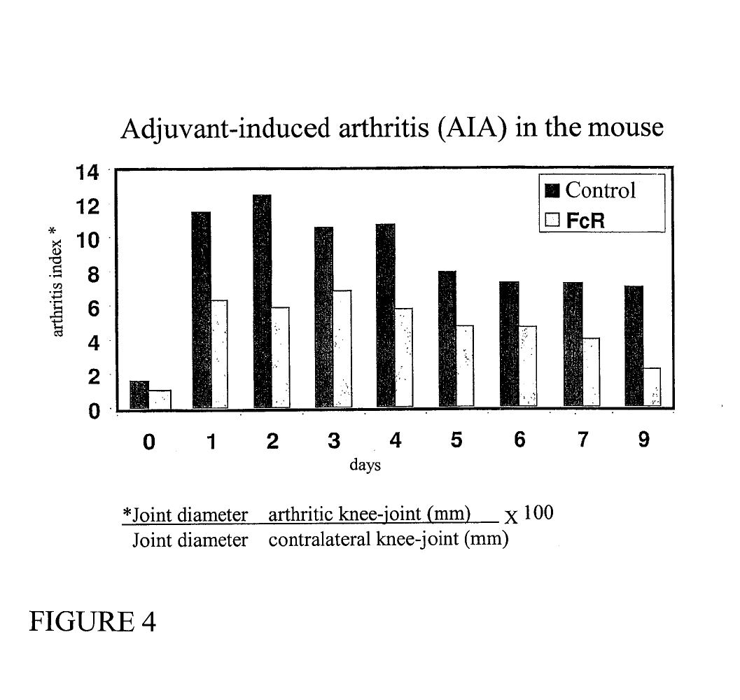

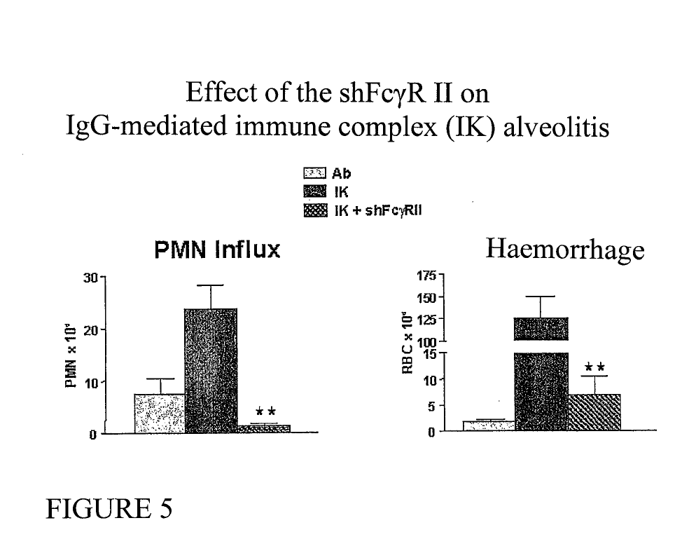

Especially, the receptor FcγR IIb with the amino acid sequence shown in SEQ ID NO. 1 and 3 has proven to be exceptionally effective when used for SLE, MS and RA. FIG. 1 shows that in the case of MS in comparison to control samples, the disease index (which is a value that indicates the severity of the disease and utilizes several defined clinical parameters for the classification) is considerably lower when FcγR IIb is administered compared to controls. In the treatment of mice which had symptoms of a lupus disease, the survival rate was also considerably increased by administering FcγR IIb according to the invention compared to a control group (FIG. 2). The increase in proteinuria which is evaluated as a measure for the deterioration of the condition of the mice was also considerably slowed down by the administration of FcγR IIb (FIG. 3). Further examples for the application of the receptors and preferably soluble FcγR IIb are rheumatoid arthritis (FIG. 4) and diseases that are associated with a high immune complex burden as shown here by immune complex-induced alveolitis as an example (FIG. 5). Overall FcγR IIb but also the other receptors of the invention has a clear positive effect on the course of diseases in which immune complexes are involved and in particular of autoimmune diseases such as multiple sclerosis, SLE or rheumatoid arthritis even when extremely low amounts of the soluble receptor are administered. It can therefore be assumed that a new mechanism of action has been discovered which could be explained by the fact that not all antibodies are eliminated by Fc receptor binding but preferentially those autoantibodies bound in immune complexes or by a new, previously unknown regulatory mechanism by which the soluble receptors intervene in the disease process.

The amounts of receptor required are also unexpectedly low in the case of FcγR III administration since it was found within the scope of the present invention that this recombinantly produced soluble FcR is surprisingly not inactive but even has a 5 to 10-fold higher ability to bind to antibodies than its natural counterpart. Thus again an extremely low dosage of FcR is sufficient and these small amounts can be used for any applications for overshooting immune reactions. Thus FcγR III is especially suitable for use as a substitute for IVIg treatment which again has the already mentioned advantages that a single injection is sufficient to administer adequate amounts of FcR. In contrast to IVIg preparations whose quality varies from batch to batch depending on the donor pool since it is isolated from human serum, the soluble FcRs described here as preferred embodiments can be produced in a constant quality. Another advantage over IVIg is the guaranteed absence of growth factor and human pathogens (e.g. HIV, hepatitis etc.) in case the FcRs are obtained from bacteria. Since it has been found that the FcRs are already effective in extremely low doses and these FcRs can also be purified particularly well and highly concentrated, their administration as an injection solution is particularly preferred. Usually a single administration of the pharmaceutical composition according to the invention is sufficient to considerably improve the symptoms and to delay acute episodes of the diseases. However, a continued application of the injections is of course also advantageous within the scope of the present invention.

Furthermore, the costs of the pharmaceutical compositions according to the invention are very low since their production by recombinant expression in for example prokaryotes is simple and results in highly-purified and highly-concentrated protein preparations. Also expression of the sFcRs in eukaryotic systems can be achieved easily and unexpensively and large amounts can be produced in high purity. Useful systems include eukaryotes with a specialized apparatus for the production of extacellular proteins, e.g. B cells. Hence the pharmaceutical compositions of the present invention can be produced at a fraction of the costs that have to be estimated for IVIg preparations.

Another subject matter of the present invention is the use of the pharmaceutical compositions according to the invention to treat overshooting immune reactions and in particular to treat multiple sclerosis, SLE, RA or to treat patients who have an elevated number of natural killer cells in their bloodstream.

As already described above it is particularly preferred in this case to use the pharmaceutical preparation according to the invention in an injectable form since the pharmaceutical composition according to the invention with its special dosage and concentration allows adequate amounts of soluble Fc receptors to also be administered in this manner. In this connection it is also particularly preferable to use the corresponding FcRs of SEQ ID NO.1 or 2 or proteins extended by wild-type sequences at the N- or/and C-terminus and especially those of SEQ ID NO. 3 or 4. The FcRs can be expressed in prokaryotes and subsequently purified and refolded according to the description of WO 00/32767. The receptors have the advantage that they can be highly concentrated and hence only small volumes are necessary to administer adequately effective amounts. Furthermore, as already described above, it was found within the scope of the present invention that the receptors apparently do not act competitively but that a new mechanism of action occurs with the result that positive effects can already be achieved by administering small amounts of the receptors. The pharmaceutical compositions according to the invention and their use for the treatment of diseases which are based on overshooting immune reactions are therefore particularly advantageous due to their dosage and ease of administration and are particularly well suited for a prolonged treatment also because of their low production costs.

The invention is elucidated by the following figures:

FIG. 1: Disease course of induced EAE in DBA/1 mice

Experimental allergic encephalomyelitis (EAE) in DBA/1 mice represents an animal model for multiple sclerosis (MS). MS-like symptoms are observed in this mouse strain (e.g. paralytic symptoms, brain lesions) after immunization with myelin oligodendrocyte glycoprotein (MOG). In each case 10 DBA/1 mice immunized in this manner were treated at 48 hour intervals with PBS (control 1), 100 μg trp-synthase from E. coli (control 2) or 100 μg soluble Fc receptor (sFcγRIIb). Symptoms of EAE were individually evaluated and plotted as a group average. The disease index (according to Abdul-Majid, K. B., Jirholt, J., Stadelmann, C., Stefferl, A., Kjellen, P., Wallstrom, E., Holmdahl, R., Lassmann, H., Olsson, T., Harris, R. A. (2000), Screening of several H-2 congenic mouse strains identified H-2(q) mice as highly susceptible to MOG-induced EAE with minimal adjuvant requirement. J. Neuroimmunol. 111: 23-33) was evaluated as follows: 0, no indication of EAE, 1, tail paralysis; 2, atony of the hind legs; 3, paralysis of the hind legs; 4, complete paralysis of the front and hind legs; 5, dying.

FIG. 2: Survival rate of NZBW/F₁ mice

NZBW/F₁ mice represent an accepted animal model for the disease systemic lupus erythematosus (SLE) (Theofilopoulos, A. N., Dixon, F. J. (1985), Murine models of systemic lupus erythematosus, Adv. Immunol. 37: 269-390). In each case 10 NZBW/F1 mice were treated subcutaneously either with PBS (control group) or with 100 μg soluble Fc receptor (sFcγRIIb) at weekly intervals. Whereas all mice were still alive 40 weeks after treatment with the Fc receptor, 70% of the control group had died after this time period.

FIG. 3: Cumulative proteinuria of NZBW/f1 mice

Proteinuria of diseased NZBW/F₁ mice as a result of developing glomerulonephritis is an important criterium for the progression of the SLE disease. The proteinuria (>0.3 g/l urine) of mice treated as stated in FIG. 1a was determined and plotted cumulatively.

FIG. 4: Disease course of adjuvant-induced arthritis (AIA) in mice

AIA represents an accepted animal model for rheumatoid arthritis. The inflammatory reaction is caused by administering antigen into a joint. The treatment is carried out intraperitoneally at weekly intervals with 100 μg soluble FcγR IIb (IP). (According to Waksman, B. H., Immune regulation in adjuvant disease and other arthritis models: relevance to pathogenesis of chronic arthritis, 2002, Scand. J. Immunol. 56(1): 12-34; Holmdahl, R., Lorentzen, J. C., Lu, S., Olofsson, P., Wester, L., Holmberg, J. & Pettersson, U., 2001, Arthritis induced in rats with non-immunogenic adjuvants as models for rheumatoid arthritis, Immunol. Rev. 184: 184-202).

FIG. 5: IgG-mediated immune complex (IC)-induced alveolitis

IC alveolitis represents an accepted animal model for inflammatory diseases that are associated with a high IC burden. For the induction mice are injected intraperitoneally (IP) with an antigen and an antibody that is directed against this antigen is administered intratracheally (IT). This causes the formation of immune complexes in the lung that result in inflammatory reactions. The infiltration of neutrophils (PMN) and haemorrhage are evaluated. The reaction in this animal model can be increased further when preformed immune complexes of antigen and antibody are administered intratracheally. Simultaneous intraperitoneal administration of 100 μg FcγR IIb almost completely suppresses the inflammatory reactions. (according to Tanoue, M., Yoshizawa, Y., Sato, T., Yano, H., Kimula, Y. & Miyamoto, K., 1993, The role of complement-derived chemotactic factors in lung injury induced by preformed immune complexes. Int. Arch. Allergy Immunol. 101(1): 47-51; Yoshizawa, Y., Tanoue, M., Yano, H., Sato, T., Ohtsuka, M., Hasegawa, S. & Kimula, Y. 1991, Sequential changes in lung injury induced by preformed immune complexes. Clin. Immunol. Immunopathol. 61(3): 376-386).

SEQ ID NO.1 shows the preferred minimal amino acid sequence of FcγR IIb which can be optionally extended at the termini.

SEQ ID NO.2 shows the preferred minimal amino acid sequence of FcγR III which can also be extended at the termini.

SEQ ID NO.3 and

SEQ ID NO. 4 show such receptor sequences extended at the termini.

SEQ ID NO. 5 shows the preferred minimal amino acid sequence of FcγRIIa which can be optionally extended at the termini.

1. Pharmaceutical composition in the form of an aqueous solution, characterized in that it contains recombinantly produced, soluble FcγRIIa or FcγRIIb from eukaryotic or in particular prokaryotic expression systems in an amount of 40 to 4000 mg, preferably 100 to 1000 mg and at a concentration of up to 50 mg/ml and optionally comprises other pharmaceutically acceptable auxiliary substances or/and excipients.

2. Pharmaceutical composition according to claim 1, characterized in that it contains the FcγRIIa FcγRIIb in an amount that allows application of 0.5 to 50 mg receptor per kg body weight of the patient.

3. Pharmaceutical composition as claimed in claim 1, characterized in that it is present in the form of an injection solution.

4. Pharmaceutical composition as claimed in claim 1, characterized in that it contains FcγRIIb according to SEQ ID NO. 1 or a form that is extended at the N-terminus or/and C-terminus with suitable sequences of the wild-type protein, preferably according to SEQ ID NO.3.

5. Pharmaceutical composition as claimed in claim 1, characterized in that it contains FcγR III according to SEQ ID NO.2 or a form that is extended at the N-terminus or/and C terminus with suitable sequences of the wild-type, preferably according to SEQ ID NO. 4.

6. Pharmaceutical composition as claimed in claim 1, characterized in that it contains FcγRIIa according to SEQ ID NO. 5.

7. Use of a pharmaceutical composition as claimed in claim 1 to combat diseases or conditions which are caused by overshooting immune reactions and a pathologically increased formation of antibodies and especially of autoantibodies.

8. The method as claimed in claim 13, characterized in that the disease is multiple sclerosis, systemic lupus erythematosus, rheumatoid arthritis or a disease that is associated with an increased number of NK cells.

9. The method as claimed in claim 13, characterized in that a pharmaceutical composition containing FcγRIIb is used.

10. Use as claimed in claim 7, characterized in that a pharmaceutical composition containing FcγR III is used.

11. The method as claimed in claim 13, characterized in that a pharmaceutical composition containing FcγRIIa is used.

12. The method as claimed in claim 13, characterized in that the pharmaceutical composition is injected.

13. Method of treating a disease or condition caused by overshooting immune reactions and a pathologically increased formation of antibodies in a patient in need of such treatment, the method of comprising administrating to the patient an effective amount of a pharmaceutical composition of claim 1.

14. The method of claim 13, wherein the antibodies are autoantibodies.

15. A pharmaceutical composition in the form of an aqueous solution, comprising a recombinantly produced, soluble FcγRIIb from a eukaryotic or prokaryotic expression system in an amount of 40 to 4000 mg, and pharmaceutically acceptable auxiliary substances or/and excipients, wherein said FcγRIIb comprises the sequence shown in SEQ ID NO:1.

16. The pharmaceutical composition according to claim 15, wherein said FcγRIIb has the sequence shown in SEQ ID NO:1 and further comprises suitable sequences at the N-terminus or/and C-terminus.

17. The pharmaceutical composition according to claim 16, wherein said suitable sequences are from the wild-type protein.

18. The pharmaceutical composition according to claim 17, wherein said FcγRIIb has the sequence shown in SEQ ID NO:3.

19. The pharmaceutical composition according to claim 15, wherein said soluble FcγRIIb is from a prokaryotic system.

20. The pharmaceutical composition according to claim 15, wherein said soluble FcγRIIb is in an amount of 100 to 1000 mg.

21. The pharmaceutical composition according to claim 15, wherein said soluble FcγRIIb is in a concentration of up to 50 mg/ml.

22. The pharmaceutical composition according to claim 15, wherein said FcγRIIb is in an amount that allows application of 0.5 to 50 mg receptor per kg body weight of the patient.

23. The pharmaceutical composition according to claim 15, wherein said pharmaceutically acceptable auxiliary substances or/and excipients are suitable for an injection solution.
